(19) United States
(12) Patent Application Publication
(10) Pub. No.: US 2024/0298908 A1
(43) Pub. Date: Sep. 12, 2024

(54) DEFORMABLE WEARABLE BIOSENSING DEVICE

(71) Applicant: Alio, Inc, Broomfield, CO (US)

(72) Inventors: Jivko Mihaylov, San Jose, CA (US); Timothy Wells, Manchester, CT (US); David Besko, Thornton, CO (US); Samit Gupta, Boulder, CO (US); Scott Davie, Boulder, CO (US); Samir Shreim, Irvine, CA (US)

(21) Appl. No.: 18/120,315

(22) Filed: Mar. 10, 2023

(51) Int. Cl. A61B 5/024; A61B 5/00

(52) U.S. Cl. CPC A61B 5/02438; A61B 5/6802

(57) ABSTRACT

A wearable biosensing device features a first housing and biosensing logic. The first housing includes a housing element and a deformable stiffening element inserted within the housing element. The housing element includes a plurality of lobes formed with a corresponding plurality of cavities. The biosensing logic is inserted into the plurality of cavities, wherein an electronics assembly is positioned within a first lobe of the plurality of lobes, a sensing assembly is positioned within a second lobe, and an interconnect for communicative coupling of the electronics assembly and the sensing assembly is positioned within a third lobe of the plurality of lobes. The forces applied by at least the first housing and the biosensing logic are balanced by the deformable stiffening element.

### FIELD

Embodiments of the disclosure relate to the field of wearable biosensing devices. More specifically, one embodiment of the disclosure relates to a biosensing device which can be mounted to a portion of the body using adhesive and is configured to improve its functionality by maintaining shape upon deformation.

### GENERAL BACKGROUND

The following description includes information that may be useful in understanding the described invention. It is not an admission that any of the information provided herein is prior art or relevant to the presently claimed invention, or that any publication specifically or implicitly referenced is prior art.

Over the last decade, there has been an increasing number of wearable biosensing devices. These devices include one or more biosensors, which collect health-related data from a user. Most of these wearable biosensing devices include a display (e.g., smart watches, Fitbits®, etc.) and are mounted on the user's wrist using a band which encircles the wrist. However, these display-based devices are costly, non-disposable, and cannot be targeted to monitor certain health characteristics, such as blood flow for example, at other location on the wearer besides the wrist area.

Adhesive-based wearable biosensing devices tend to be disposable, where the conventional adhesive used to attach this type of biosensing device to a wearer's body must be biocompatible and hypoallergenic, and perhaps sterilized. The reliability of data collected from these types of biosensing devices depends on achieving and maintaining a consistent coupling of the biosensing device to the wearer's body. Many of the adhesives which are capable of providing a consistent coupling of the biosensing device with the body suffer from the following tradeoff-either (1) the adhesive degrades quickly over time, especially when the biosensing device is placed on a curved surface, leading to a poor coupling between the device and the skin or (2) the adhesive is so strong that the biosensing device is uncomfortable to wear as it may prohibit or cause pain for certain movement. What is needed is an adhesive-based wearable biosensing device that is configured to maintain its shape upon deformation by counteracting the potential energy associated with the housing and other components attempting to return the wearable biosensing device back to its non-deformed, linear state. Otherwise, as currently observed, wearable biosensing devices tend to quickly become decoupled when attached to curved surfaces and adversely affect data collection that is critical in the monitoring of the wearer's health.

### BRIEF DESCRIPTION OF THE DRAWINGS

Embodiments of the invention are illustrated by way of example and not by way of limitation in the figures of the accompanying drawings, in which like references indicate similar elements and in which:

FIG. 1 is a top view of an exemplary embodiment of a wearable biosensing device

FIG. 2 is an exploded view of an exemplary embodiment of the wearable biosensing device of FIG. 1, which includes a first housing, biosensing logic, a second housing, and an adhesive layer.

FIG. 3 is an exploded view of the exemplary embodiment of the first housing for the wearable biosensing device of FIG. 2.

FIG. 4A is a cross-sectional view of the first housing for the wearable biosensing device of FIG. 2 including a molded housing element and a deformable stiffening element integrated into the housing element.

FIG. 4B is a cut-out, top-perspective view of an exemplary embodiment of the first housing for the wearable biosensing device of FIG. 2 illustrating positioning of the stiffening element relative to lobes of the housing element.

FIG. 4C is a bottom planar view of an exemplary embodiment of the first housing for the wearable biosensing device of FIG. 2.

FIG. 5 is a perspective view of an exemplary embodiment of the biosensing logic for the wearable biosensing device of FIG. 2.

FIG. 6 is a perspective view of an exemplary embodiment of the second housing for the wearable biosensing device of FIG. 2.

FIG. 7 is a bottom perspective view of the first housing for the wearable biosensing device of FIG. 2.

FIG. 8 is a bottom perspective view of the first housing deploying the biosensing logic as shown in FIG. 2 with the biosensing logic of FIG. 5 deployed within cavities formed within the first housing and an adhesive attached to interior, recessed surfaces of the first housing.

FIG. 9 is a bottom perspective view of the wearable biosensing device illustrating the second housing being coupled to the first housing to encapsulate the biosensing logic.

FIG. 10 is a bottom-side perspective view of the wearable biosensing device illustrating an application of an adhesive to being applied to bottom surfaces of both the first housing and the second housing.

FIG. 11 is a bottom, cut-away view of a wearable biosensing device including the first housing, the biosensing logic, the second housing highlighting a central window for positioning of optical sensing elements deployed within a sensing assembly positioned above the central window.

FIGS. 12A-12B are illustrative embodiments of functional representations of the configuration of the wearable biosensing device with respect to the resistance of forces applied to the device.

### DETAILED DESCRIPTION

Embodiments of the present disclosure generally relate to a wearable biosensing device that features biosensing logic deployed within a housing and attached to a wearer through adhesive. The wearable biosensing device is particularly advantageous for monitoring characteristics and operability of a vessel (e.g., an artery, a vein, or an arteriovenous (AV) fistula) by collecting information associated with physiological properties of that vessel and/or the biological fluid propagating therethrough (e.g., flow, fluid composition, etc.). This information is especially useful to monitor the health of a patient, especially dialysis patients, and may be used to generate an alert signifying a detected health event that is being (or could be) experienced by the wearer of the wearable biosensing device. The wearable biosensing device may include multiple sensors (e.g., optical, audio, temperature, bioimpedance, electrocardiography etc.), where each sensor or sensors is configured to detect/monitor a physiological property, convert the monitored property into an electrical signal, which is subsequently converted to a data representation of the monitored property indicated by the electrical signal. The data representation enables remote monitoring.

As an illustrative example, the wearable biosensing device (with remote monitoring) may be mounted on or proximate to a vessel, such as an artery, a vein, or an arteriovenous (AV) fistula as disclosed in U.S. Pat. Nos. 11,045,123 and 11,406,274, the contents of both of which are incorporated by reference herein. The AV fistula is a surgical connection made between an artery and a vein, created by a vascular specialist. The AV fistula facilitates efficient dialysis than a line due to quicker blood flow during a dialysis session. Normally, the AV fistula is typically located in your arm, however, if necessary, it can be placed in the leg. Other uses for the wearable biosensing device include mounting on the chest for monitoring cardiac functions or on the abdomen for prenatal or intestinal monitoring.

Herein, the wearable biosensing device is configured to maintain shape upon deformation, such as when applied to a surface of high curvature for example (e.g., bending of the housing associated with the wearable biosensing device by an acute or obtuse angle). This configuration involves a device architecture that supports deformation of the biosensing device from a first (flat) orientation to a second orientation and retention of the biosensing device within the second orientation. The second orientation may feature a single deviation from its flat orientation, such as a selected curvature for mounting on an inner arm region of the wearer. The second orientation may feature multiple deviations from its flat orientation (e.g., curved, angular deviation lateral and/or elevational, etc.) for attachment to any three-dimensional shape to account for skin/body deformities or irregularities of the wearer.

Stated differently, the wearable biosensing device is constructed to maintain a conformal or near conformal geometry, which is achieved by mitigating (or counteracting) elastic potential energy stored in the housing and/or other device components (e.g., normal forces incident on the adhesive layer) that encourage return of the wearable biosensing device back to its original, linear orientation. The device's deformable property provides an improvement in user experience and improves overall operability of the biosensing device in monitoring properties associated with a targeted vessel, which will increase compliance in wearing the biosensing device.

According to one embodiment of the disclosure, the deformable property of the wearable biosensing device may be accomplished by integrating one or more deformable stiffening elements (hereinafter, “deformable stiffening element”) as part of the housing. As an illustrative example, the deformable stiffening element may be incorporated within a first (upper) portion of the housing for the wearable biosensing device (hereinafter, “first housing”), which features lobes and corresponding cavities that are sealed by a second housing. The first housing may be made of a flexible material (e.g., a polymer such as silicone, plastic, etc.) formed with a selected structure through a molding process, where the deformable stiffening element is embedded as part of the molded first housing.

As another illustrative embodiment, the deformable stiffening element may be integrated within the second housing or may be attached to the first housing after molding has completed or placed into a pocket or continuous recessed portion formed within the first housing, where the pocket is created to house the deformable stiffening element. In general, the deformable stiffening element may be situated within any of a number of portions forming the first housing and/or the second housing.

In accordance with another embodiment of the disclosure, the biosensing logic may include a deformable stiffening element attached to certain assemblies and/or interconnects forming the biosensing logic. Additionally, or in the alternative, the interconnects associated with the biosensing logic may be of a prescribed thickness and/or material (e.g., metal, conductive material, etc.) so as to operate as a deformable stiffening element by counteracting the potential energy associated with the deformed housing and/or components needed to return the wearable biosensing device back to its substantially linear orientation. Functionally, the stiffening element is arranged in parallel with the elastic elements, namely the first and second housings (e.g., the silicone preform) and the adhesive layer. Lastly, as yet another illustrative embodiment, the material used in construction of the first (or second) housing may be configured to maintain shape upon deformation.

As described below, the geometry of the deformable stiffening element may be chosen to provide consistent deformation across its length and width. Conversely, the geometry of the deformable stiffening element may be designed to encourage bending at specific locations and in specific directions. One non-limiting example would be thickness variations specifically chosen to promote bending at particular locations. As a result, the housing is designed to encourage bending in specific locations, where the properties of the housing and the deformable stiffening element work together to encourage bending in specific locations. As a logical representation, the deformable stiffening element operates in parallel with the elastic (housing-based) elements

As shown and described below, the deformable stiffening element may be positioned proximate to lobes formed within the first housing, where both a first (front) lengthwise edge and a second (rear) lengthwise edge of the deformable stiffening element are curved inwardly toward a central area of the first housing. This inward curvature of the deformable stiffening element and the top housing forms a recessed, central area so that the width is less than the normal spacing for cannulation needles needed during dialysis. Also, the inward curvature may promote bending at locations between the central area and distal (end) areas, which may cause the wearable biosensing device to remain substantially linear toward the sensing assembly, but allow for the deformation of the wearable biosensing device near the distal areas to improve retention of the device on body parts with high curvature (e.g., forearm, neck, various leg areas, etc.). The selected structure of the deformable stiffening element may be complementary in shape to the architecture of its housing in which it is deployed (e.g., first housing).

In some embodiments, the deformable stiffening element is made of a semi-rigid material such as certain types of metal (e.g., aluminum, certain grades of steel, copper, spring steel, etc.) or even certain plastic materials. The deformable stiffening element may have a homogenous structure, where this deformable stiffening element is made of a single material. For example, in one illustrative embodiment, the deformable material comprises a relatively low yield stress and comparatively high elastic modulus (e.g., 1100 series aluminum) such that deformation occurs with little to no return to previous geometry. A stiffener element comprising such material (e.g., 1100 series aluminum) would be subject to very little to no hysteresis to enable the wearable to conform to the subject geometry with little resistance while maintaining the advantages of the disclosed invention. Alternatively, the deformable stiffening element may be made of a plurality of different materials chosen to achieve specific design goals. For example, as one illustrative example, the deformable stiffening element may feature a bi-metal construction, where certain areas with a first type of metal may deform more easily than areas with a second type of metal (e.g., different responses to temperature change, different flexural strength, etc.). As another illustrative embodiment, the deformable stiffening element may include distinct materials chosen based on yield points to achieve specific conformal geometry. In another alternative, the stiffener comprises a single material where geometry is used to adjust the flexural strength and or elastic modulus to encourage bending in particular areas and discourage bending in other areas.

More specifically, according to one embodiment of the disclosure, the wearable biosensing device features a first (top) housing embedded with the deformable stiffening element and a second (bottom) housing coupled to the first housing to encapsulate biosensing logic therein. The biosensing logic may include, but is not limited to (i) a processing, storage, and communication (PSC) assembly, (ii) a sensing assembly, and (iii) a power assembly. The PSC assembly is arranged to reside within a first cavity formed by a first lobe molded as part of the first housing. Similarly, the sensing assembly is arranged to reside within a second cavity formed by a second lobe molded as part of the first housing, and the power assembly is arranged to reside within a third cavity formed by a third lobe. The second housing may include an opening to be aligned with sensors, such as optical sensors to emit light signals and capture returned light (by reflection, scattering, or refraction).

As the sensing assembly is positioned between the PSC assembly and the power assembly, interconnects are needed to supply power and maintain communications between these assemblies. As described below, the first housing further includes a fourth lobe and a fifth lobe. The fourth lobe provides a channel between the first lobe and the second lobe for encapsulating a first interconnect extending between the sensing assembly and the PSC assembly. Similarly, the fifth lobe provides a channel between the second lobe and the third lobe for encapsulating a second interconnect extending between the sensing assembly and the power assembly.

### I. Terminology

In the following description, certain terminology is used to describe aspects of the invention. The terms “logic” or “assembly” are representative of hardware, firmware, and/or software that is configured to perform one or more functions. As hardware, the logic (or assembly) may include circuitry associated with data processing, data storage and/or data communications. Examples of such circuitry may include, but are not limited or restricted to a processor, a programmable gate array, a microcontroller, an application specific integrated circuit, wireless receiver, transmitter and/or transceiver circuitry, sensors, semiconductor memory, or combinatorial logic.

Alternatively, or in combination with the hardware circuitry described above, the logic (or assembly) may include software in the form of one or more software modules, which may be configured to operate as its counterpart circuitry. For instance, a software module may be a software instance that operates as a processor, namely a virtual processor whose underlying operations is based on a physical processor such as virtual processor instances for Microsoft® Azure® or Google® Cloud Services platform or an EC2 instance within the Amazon® AWS infrastructure, for example.

Additionally, a software module may include an executable application, a daemon application, an application programming interface (API), a subroutine, a function, a procedure, an applet, a servlet, a routine, source code, a shared library/dynamic load library, or even one or more instructions. The software module(s) may be stored in any type of a suitable non-transitory storage medium, or transitory storage medium (e.g., electrical, optical, acoustical, or other form of propagated signals such as carrier waves, infrared signals, or digital signals). Examples of non-transitory storage medium may include, but are not limited or restricted to a programmable circuit; a semiconductor memory; non-persistent storage such as volatile memory (e.g., any type of random access memory “RAM”); persistent storage such as non-volatile memory (e.g., read-only memory “ROM”, power-backed RAM, flash memory, phase-change memory, etc.), a solid-state drive, a hard disk drive, an optical disc drive, a portable memory device, or cloud-based storage (e.g., AWS S3 storage, etc.), as described below. As firmware, the logic (or assembly) may be stored in persistent storage.

The terms “member” and “element” may be construed as a physical feature. The term “cavity” may be constructed as a recessed area within a housing element. As a result, the cavity operates as a chamber that is configured to secure, maintain, and protect components including electronic and/or sensor components.

The term “attach” and other tenses of the term (attached, attaching, etc.) may be construed as physically connecting a first member to a second member. Additionally, the term “deformable” with respect to a member or portion of a member may be construed as the member being configured to resist, at least partially, bending or deformation from its original orientation (state), but once deformed, remains in its deformed orientation.

The term “interconnect” may be construed as a physical or logical communication path between two or more components such as a pair of assemblies. For instance, as a physical communication path, wired interconnects in the form of electrical wiring, optical fiber, cable, and/or bus trace. As a logical communication path, the interconnect may be a wireless channel using short range signaling (e.g., Bluetooth™) or longer range signaling (e.g., infrared, radio frequency “RF” or the like).

Finally, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, functions, steps, or acts are in some way inherently mutually exclusive.

As this invention is susceptible to embodiments of many different forms, it is intended that the present disclosure is to be considered as an example of the principles of the invention and not intended to limit the invention to the specific embodiments shown and described.

### II. Device Attachment Scheme

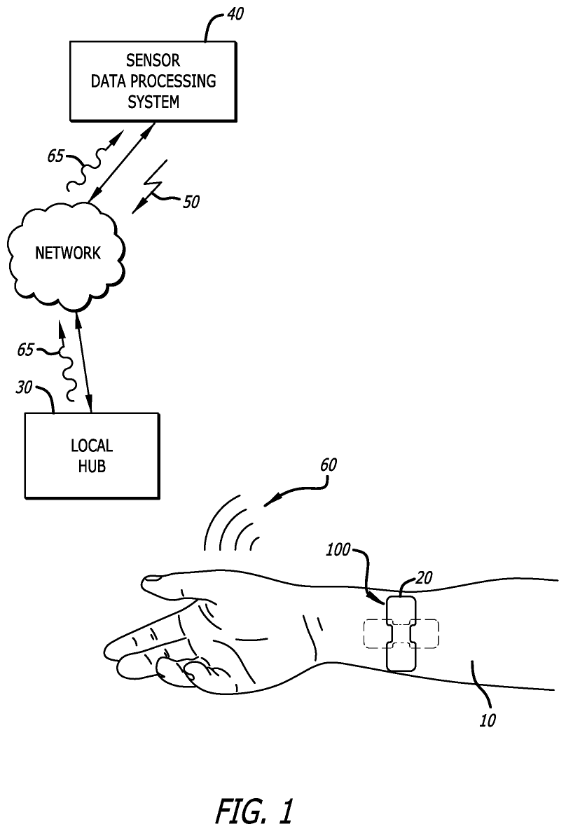

Referring to FIG. 1, a top view of an example of a wearable biosensing device 100 attached to a patient's arm 10. The wearable biosensing device 100 is intended to be worn over a vessel (e.g., an artery, a vein, or an arteriovenous (AV) fistula) 20 and configured to direct collected information to a remotely located, sensory data processing system 40. For this embodiment, the wearable biosensing device 100 is configured to monitor properties (e.g., characteristics and operability) of AV fistula 20 by collecting information associated with the AV fistula 20 and the biological fluid propagating therethrough (e.g., flow, fluid composition inclusive of hemoglobin levels, etc.). The collected information may be used for remote monitoring, where the sensory data processing system 40 is configured to determine if a health event exists that warrants generation and transmission of an alert 50 to the wearer or an individual involved with the care of the wearer.

More specifically, the remote monitoring may involve transmission of the collected information to a local hub 30. The “local hub” 30 constitutes logic (e.g., a device, an application, etc.) that converts a first data representation 60 of the collected information of a first transmission protocol (e.g., Bluetooth™ or other short distance (wireless) transmission protocol) into a second data representation 65 of a second transmission protocol (e.g., cellular, WiFi™ communications or other long distance (wireless) transmission protocol) and routes the second data representation 65 to the sensory data processing system 40. The sensory data processing system 40 may include an alerting system (not shown), which generates and sends the alert (notification) 50 upon detecting an occurring (or potential) health event that requires attention by a doctor or another specified person (including the wearer) responsible for addressing any occurring (or potential) health event. The alert 50 may be sent using any suitable communications system such as, for example, notification via an electronic mail (e-mail) message, a notification on a web-site, a text message, or any other suitable signaling mechanism.

As an illustrative example, the AV fistula 20 may become occluded over time during use and prevent the patient from receiving dialysis treatment. The blockage can typically either be acute from thrombosis or occur over time through stenosis. If blockage is detected early enough, there are treatments that can unclog the AV fistula 20 (e.g., thrombectomy, angioplasty) while pre-serving access. If a thrombosis forms, clinicians must intervene prior to the thrombus hardening (typically occurs within 48-72 hours) order to successfully treat the patient and preserve the access. If left untreated, the access may need to be replaced which leads to 4-12 weeks of catheter-based dialysis in the patient's treatment. Hence, the monitoring of the AV fistula 20 is directed to its operability, where without such monitoring, the patient will need to subject herself or himself to invasive medical procedures.

### III. General Device Architecture

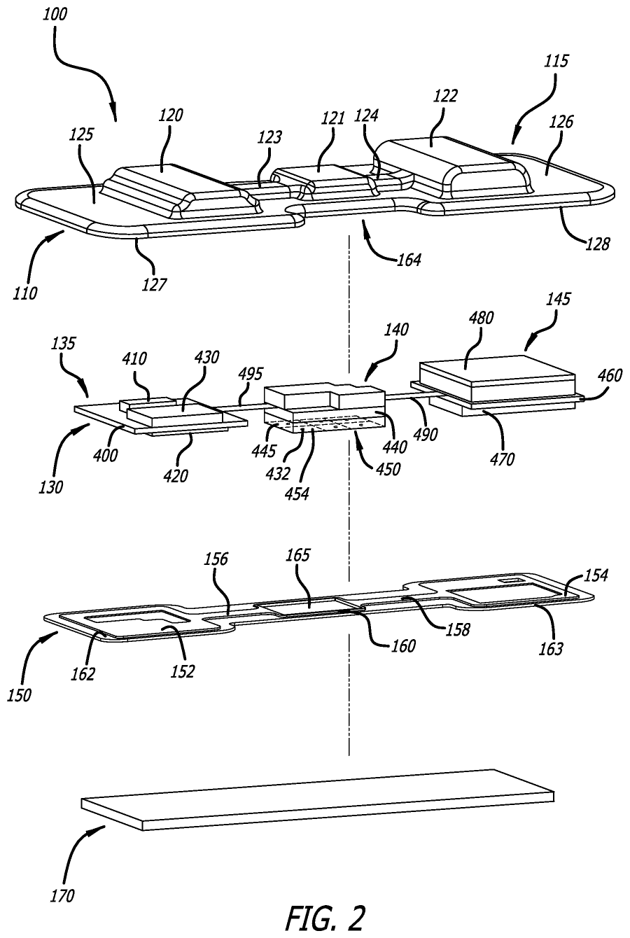

Referring to FIG. 2, an exploded view of an exemplary embodiment of the wearable biosensing device 100 is shown. For this embodiment, the wearable biosensing device 100 includes a first (top) housing 110, biosensing logic 130, a second (bottom) housing 150, and an adhesive layer 170. The first housing 110 features multiple (e.g., two or more) lobes 115 formed in the first housing 110. The first housing 110 may be made of a flexible, water-impervious material (e.g., a polymer such as silicone, plastic, etc.) through a molding process, where the lobes 115 provide cavities for housing the biosensing logic 130.

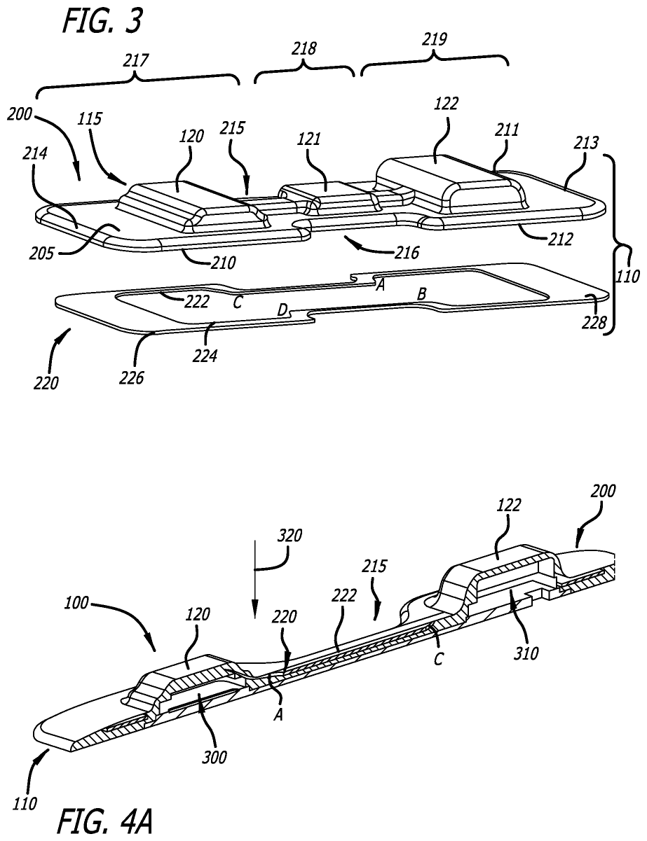

As an illustrative example, according to one embodiment of the disclosure, the lobes 115 are positioned in a linear orientation, with a first plurality of lobes (e.g., first, second and third lobes 120-122) interconnected by a second plurality of lobes (e.g., fourth and fifth lobes 123-124). The first housing 110 further includes a first end portion 125 that extends distally from the first lobe 120 and a second end 126 that extends from the third lobe 122. Although shown in FIG. 3, a deformable stiffening element 220 may be added to the first housing 110 (e.g., integrated within the molded structure forming the first housing 110) to provide controlled rigidity of the first housing 110 and thereby counteract potential energy maintained in the first housing 110 after deformation from a first (substantially linear) orientation into a second (curved or angular) orientation. Hence, the wearable biosensing device 100 may be placed into the second orientation and remains in this orientation.

The biosensing logic 130 includes an electronics assembly 135, a sensing assembly 140, and a power assembly 145. According to one embodiment of the disclosure, as shown in FIGS. 2&5, the electronics assembly 135 includes a substrate 400, processing logic 410, memory logic 420, and communications logic 430. Collectively, logic of the electronics assembly 135 operates to conduct analysis of data gathered by the sensing assembly 140, stores the raw and/or analyzed data, and communicates, wirelessly or through a wired connection, the raw data and/or the analyzed results to a device (e.g., local hub 30 of FIG. 1) remotely located from the wearable biosensing device 100.

As shown in FIGS. 2&4, the sensing assembly 140 includes a substrate 440, an optional, shielding member 445, and one or more optical sensors 450, which are configured to emit light and/or detect reflected or refracted light. The optical sensors 450 may include photo-plethysmograph (PPG) sensors, which include a light sourcing member (e.g., light emitting diodes of different wavelength ranges such as wavelengths of around 532 nanometers (nms) ranging between 520-540 nm, 655 nm ranging between 645-665 nm, 940 nm ranging between 930-950 nm, etc.), light detecting member (e.g., photodetectors to capture the reflected or refracted light), and amplifiers to amplify the received results. Stated differently, according to one embodiment of the disclosure, a plurality of light emitting diodes (LEDs) 452 with different light wavelengths (e.g., color spectrums) operating in combination with complementary photodetectors 454 may be used to monitor fluid flow, vessel characteristics (e.g., depth, diameter, etc.), oxygenation levels of fluid flowing through the vessel, hemoglobin levels, or the like.

Coupled to the PSC assembly 135 and the power assembly 145 via interconnects 490 and 495, the sensing assembly 140 may be mounted on or positioned proximate to the vessel 20 of FIG. 1. As a result, the optical sensors 450 may be used to obtain different measurements of properties of the vessel 20 or biological fluid characteristics within the vessel 20 and provide this data to the electronics assembly 135 for analysis. The optical sensors 450 may be arranged in a linear arrangement (as shown) or a circular arrangement with a light sourcing member being positioned centrally and light detecting members distributed radially from the central light sourcing member.

Besides the optical sensors 450, the sensing assembly 140 may be configured to include audio sensing logic (e.g., microphone), logic to sense bio-impedance, and/or electrocardiogram (EKG) logic.

The power assembly 145 includes a substrate 460, power management logic 470, and power supply logic 480. Power supply logic 480 is configured to provide power to both the components within the sensing assembly 140 as well as the electronics assembly 135. The power management logic 470 is configured to control the distribution of power (e.g., amount, intermittent release, or duration), including disabling of power when the wearing biosensing device 100 is not installed or detached to the wearer to avoid false data collection. The substrate 440 of the sensing assembly 140 may include hardwired traces (power layers) for routing of power from the power supply assembly 145 to the PSC assembly 135.

Referring back to FIG. 2, the second housing 150 is configured to rest within and adhere to a corresponding recessed region (not shown) accessible from an underside of the first housing 110. The second housing 150 includes a first raised structure 152 and a second raised structure 154 with an opening 165 positioned between these raised structures 152 and 154. Third and fourth raised structures 156 and 158 are coupled to a raised structure 160 that surrounds the opening 165. Each of these raised structures 152, 154, 156, 158 and 160 are formed on a top side 162 of the second housing 150 to provide a force against components of the assemblies 135, 140 and 145 as well as the interconnects 490 and 495 of the biosensing logic 130 when the second housing 150 is attached to the first housing 110. These raised structures 152, 154, 156, 158 and 160 are configured to mitigate movement of components (e.g., logic and interconnects) when the components are placed within the first housing 110.

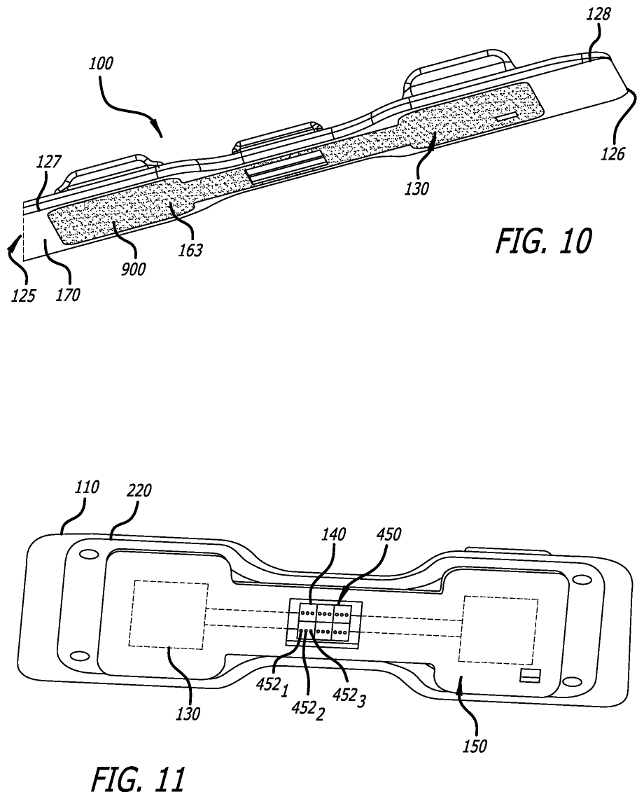

Additionally, the second housing 150 features a bottom surface 163 that, when the second housing 150 is attached to the first housing 110, is generally coplanar with bottom surfaces 127 and 128 of the first and second end portions 125 and 126, respectively. Also, the bottom surface 163 of the second housing 150 may be coplanar to bottom surfaces 164 of at least areas within the first housing 110 occupied by the deformable stiffening element 220 of FIG. 3 (see FIG. 10). As a result, according to one embodiment of the disclosure, the entire bottom surface 163 of the second housing 150 and certain areas of the bottom surface 164 of the housing 110 are substantially coplanar.

Referring still to FIG. 2, the adhesive layer 170 is applied to the bottom surfaces 163 and 164 of both the second housing 150 and areas of the first housing 110 that is still visible after attachment of the second housing 150 to the first housing 110. The adhesive layer 170 is adapted to attach to a surface of a wearer's skin and remain attached thereto.

Referring now to FIG. 3, an exploded view of the exemplary embodiment of the first housing 110 for the wearable biosensing device 100 of FIG. 2 is shown. The first housing 110 includes a housing element 200 and a deformable stiffening element 220. Although shown separately for clarity, the deformable stiffening element 220 may be inserted into a base layer 205 of the housing element 200 so as to partially surround the lobes 115 extended from the base 205. This may occur during the molding process in which the deformable stiffening element 220 would be embedded (or integrated) into the molded housing element 200. Alternatively, the deformable stiffening element 220 may be inserted into a prescribed recess portion (or pocket) formed in the housing element 200 after the molding process.

Herein, according to one embodiment of the disclosure, the base member 205 includes a perimeter 210, featuring a first longitudinal edge 211, a second longitudinal edge 212, a first latitudinal edge 213, and a second latitudinal edge 214. The first and second longitudinal edges 211 and 212 are arranged in a mirrored configuration, where both central segments 215 and 216 of the longitudinal edges 211 and 212 are recessed inwardly to account for the spacing for insertion of cannulation needles as needed for dialysis. Hence, a first region 217 of the base 205, which includes the first lobe 120, is larger in size (e.g., area and/or dimension) than a second region 218 that includes the second lobe 121. Similarly, a third region 219 of the base 205, which includes the third lobe 122, is also larger in size than the second region area 218 of the base 205.

The deformable stiffening element 220 includes a first longitudinal frame member 222, a second longitudinal frame member 224, a first lateral frame member 226, and a second lateral frame member 228. The first and second longitudinal frame members 222 and 224 of the deformable stiffening element 220 are similarly configured as the first and second longitudinal edges 211 and 212 of the base 205, respectively. Herein, the first and second longitudinal edges 222 and 224 of the deformable stiffening element 220 are configured to abut against a portion of the periphery of each of these lobes 120-125. A first end 226 of the deformable stiffening element 220 is sized with a width and length that is lesser in size than the width and length of a portion of the first region 217 extending distally from an outer edge of the first lobe 120. The second end 228 of the deformable stiffening element 220 is also sized with a width and length that is lesser in size than the corresponding width and length of a portion of the third region 219 extending distally from an outer edge of the third lobe 122.

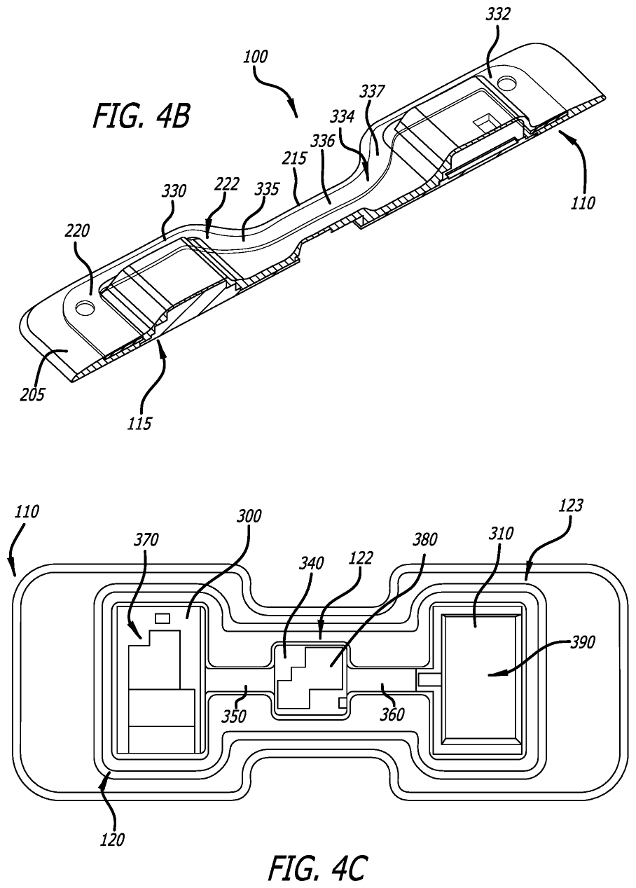

Referring now to FIG. 4A, a cross-sectional view of the first housing 110 for the wearing biosensing device 100 of FIG. 2, which includes the housing element 200 and the deformed stiffening element 220 integrated into the housing element 200, is shown. Herein, portions of a longitudinal edge (e.g., the first longitudinal edge 222) are illustrated, where the first longitudinal edge 222 is arranged to circumvent outer edges of at least the first lobe 120 and the third lobe 122 of the housing element 200. For example, the first longitudinal edge 222 is arranged to circumvent the outer edges for all of the lobes 120-125 of FIG. 2. As a result, the deformable stiffening element 220 does not extend into cavities 300 and 310 formed by lobes 120 and 122, respectively. Rather, the first longitudinal edge 222 partially surrounds and extends along an outer periphery of these lobes 120-125. The same configuration would be applicable for the second longitudinal edge 224 of the deformable stiffening element 220.

The presence of the deformable stiffening element 220 enables the wearable biosensing device 100 to be adjusted (deformed) from a substantially linear orientation (original state) into a curved orientation (deformed state). For this embodiment, the deformable stiffening element 220 may be made of a semi-rigid material such as certain types of metal (e.g., aluminum, certain grades of steel, cooper, spring steel, etc.) or even certain plastic materials. The deformable stiffening element 220 may have a homogenous structure, where this deformable stiffening element is made of a single material. Alternatively, the deformable stiffening element 220 may be made of a plurality of different materials chosen to achieve specific design goals.

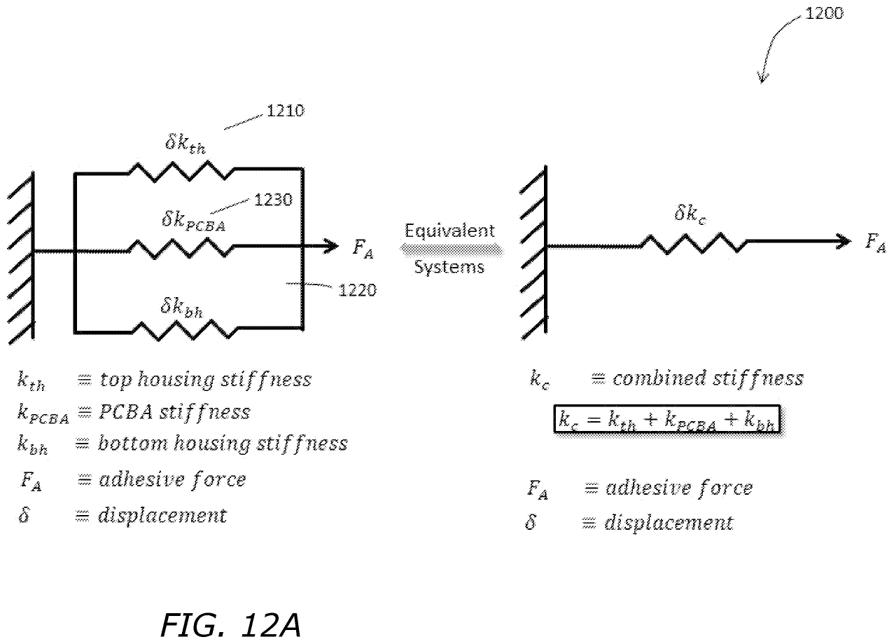

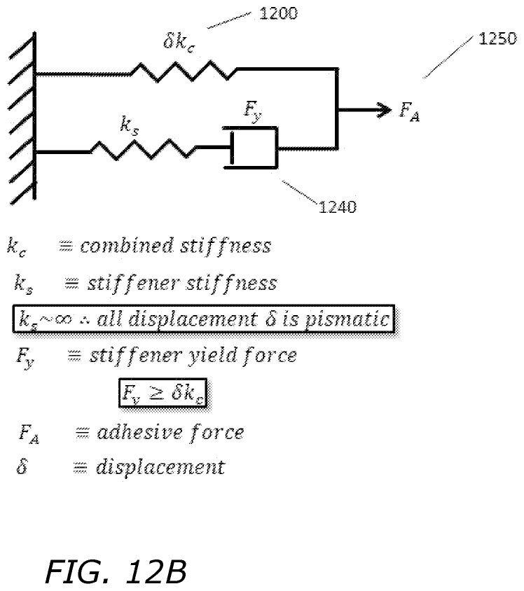

The deformable stiffening element 220 is arranged with bending locations A-D (see also FIG. 3) that are most susceptible to bending caused by downward forces 320. For example, as one illustrative example, the deformable stiffening element 220 may feature a bi-metal construction, where certain areas with a first type of metal may deform more easily than a second metal type (e.g., different flexibility due to temperature change, different flexural strength, etc.). As another illustrative embodiment, the deformable stiffening element may include distinct materials, or a selected construction having yield points (A-D) to achieve specific conformal geometry and counteract potential energy of the housing attempting to return to its linear orientation as functionally illustrated in FIGS. 12A-12B. As a result, upon deformation, the wearable biosensing device 100 tends to remain in its deformed orientation (bent or angular) until additional forces are applied to alter this deformed orientation.

Referring to FIG. 4B, a cutout, top perspective view of an exemplary embodiment of the first housing 110 for the wearable biosensing device 100 FIG. 2 is shown, where the deformable stiffening element 220 and its positioning relative to the lobes 115 is further shown. Herein, the first longitudinal edge 222 of the deformable stiffening element 220 is shown in which a first segment 330 of the longitudinal edge member 222 is configured to partially surround the first lobe 120 and a second segment 332 of the first longitudinal edge 222 partially surrounds the third lobe 122. The first longitudinal edge 222 further includes a third segment 334 that is configured to mimic the curvature of the central segment 215 of the base 205, and thus, the third segment 334 features an angular first subsegment 335, a linear second subsegment 336 and an angular third subsegment 337. The second subsegment 336 is positioned adjacent to the second lobe 121 while the first subsegment 335 and the third subsegment 337 are positioned adjacent to the first lobe 120 and the third lobe 122, respectively.

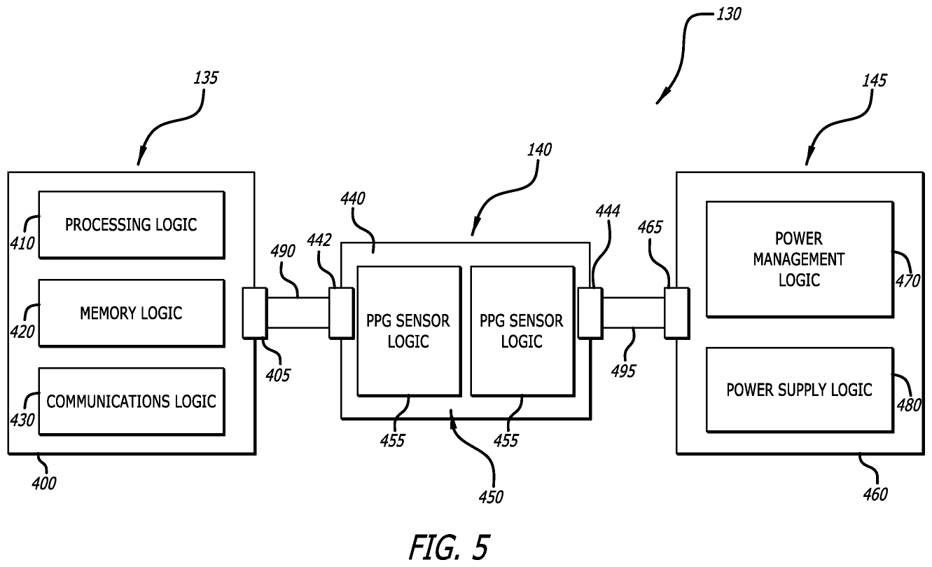

Referring now to FIG. 4C, a bottom planar view of an exemplary embodiment of the first housing 110 for the wearable biosensing device 100 of FIG. 2 is shown. The first housing 110 is formed with the first cavity 300 associated with the first lobe 120, a second cavity 340 associated with the second lobe 121, and the third cavity 310 associated with the third lobe 123. The first cavity 300 includes additional sub-cavities 370 to provide additional clearance for components that are placed within the cavity 300 such as, for example, components on the substrate 400 (e.g., circuit board) of FIG. 5, which may include, but is not limited or restricted to the processor logic 410, the memory logic 420, and the communication logic 430. The sub-cavities 370 ensure that each of the components of the PSC assembly 135 and the substrate 400 are oriented adjacent to an inner surface of the first cavity 300. Similarly, sub-cavities 380 and 390 are positioned on inner surfaces of the second cavity 340 and third cavity 310, respectively.

Adjacent to the first cavity 300, a fourth cavity 350 is formed to maintain the first interconnect 490 to be disposed therein. The first interconnect 490 supports a routing of data and/or power between the electronics assembly 135 to the sensing assembly 140. Similarly, adjacent and interposed between the second and third cavities 310 and 340, a fifth cavity 360 is formed to maintain the second interconnect 495. The second interconnect 495 supports a routing of power between the power assembly 145 and the sensing assembly 140, which operates as an intermediary for continued propagation of power to the electronics assembly 135.

Referring now to FIG. 5, a perspective view of an exemplary embodiment of the biosensing logic for the wearable biosensing device of FIG. 2. Herein, the biosensing logic 130 includes the electronics assembly 130, the sensing assembly 140, and the power assembly 145, all of which are communicatively coupled through the first interconnect 490, and the second interconnect 495. According to this embodiment of the disclosure, the electronics assembly 130 includes the substrate 400 upon which the processing logic 410, the memory logic 420, and the communication logic 430 are mounted. The processing logic 410 is configured to process data that is sensed and collected by the sensing assembly 140 for processing and subsequent output to an external computing device via the communication logic 430. The memory logic 420 may be configured to provide storage of sensed (raw) data collected by the sensing assembly 140 as well as results of the processing of the sensed data by the processing logic 410.

As further shown in FIG. 5, the sensing assembly 140 includes the substrate 440 and the optical sensor(s) 450, which are configured to emit light and/or detect reflected or refracted light. The optical sensor(s) 450 may include one or more photo-plethysmograph (PPG) sensor logic 455. Herein, according to one embodiment of the disclosure, each PPG sensor logic 455 includes one or more light sources and their corresponding light detectors. As an illustrative example, each light source may correspond to a light emitting diode (LED) of a certain color spectrum while its corresponding light detector may include a photodiode to detect light having a light spectrum of a counterpart LED reflecting from the wearer's skin and/or vessels in close correspondence to the skin surface of the wearer.

Additionally, the sensing assembly 140 further includes a first interface 442 and a second interface 444. The first interface 442 is connected to the first interconnect 490, which is coupled to an interface 405 situated on the substrate 400. The interface 405 operates as an input/output for receiving power from the power assembly 145 via the first interface/interconnect 442/490, where the received power is subsequent routed over power traces on the substrate 400. The interface 405 further operates as an input/output for receiving collected information from the sensing assembly 140 via the first interface/interconnect 442/490, where the received information is subsequently routed over data traces on the substrate 400 to the processing logic 410 and/or memory logic 420.

The second interface 444 is connected to the second interconnect 495, which is coupled to an interface 465 situated on the substrate 460. The second interface 444 operates as an input/output for receiving power over the second interconnect 495, where a portion of the supplied power is utilized by components mounted on the sensing assembly 140 and another portion of the power is provided via the first interface 442 to the PSC assembly 135 as described above. The substrate 450 includes power traces for subsequently routing of a portion of the received power to the first interface 442 for transmission to the electronics assembly 135 over the first interconnect 490.

The power assembly 145 includes the substrate 460, the power management logic 470, and the power supply logic 480. The power supply logic 480 is configured to provide power to both the components within the sensing assembly 140 as well as the electronics assembly 135. The power management logic 470 is configured to control the distribution of power (e.g., amount, timing of power releases such as throttled or intermittent release, duration, etc.), including disabling of power when the wearable biosensing device 100 is not installed or detached to the wearer to avoid false data collection.

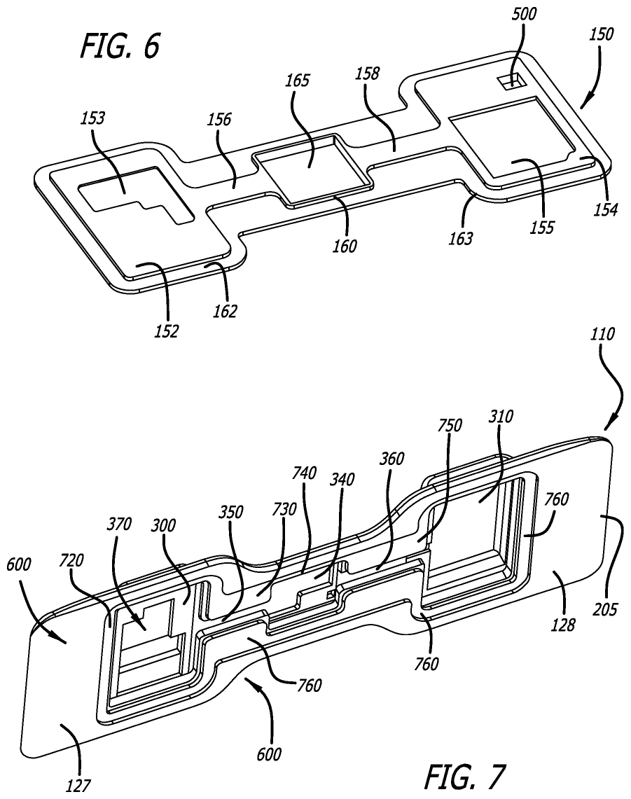

Referring to FIG. 6, a perspective view of an exemplary embodiment of the second housing 150 for the wearable biosensing device 100 of FIG. 2 is shown. Herein, the second housing 150 includes the opening 165 positioned between the first raised structure 152 and the second raised structure 154. The first raised structure 152 is sized for partial insertion into and sealing of the first cavity 300 with the electronics assembly 135 installed therein. The first raised structure 152 may include one or more recesses 153 correspond to a shape of a component mounted on a bottom surface of the substrate 400 of FIG. 2.

Similarly, the second raised structure 154 is sized for partial insertion into and sealing of the second cavity 310 with the power assembly 145 installed therein. Additionally, the second raised structure 154 may include one or more recesses 155 correspond to a shape of a component mounted on a bottom surface of the substrate 460 of the power assembly 145 (see FIG. 2). The second raised structure 154 may also include a cut-out 500 that provides a temperature sensing element (located on the substrate 460) with access the skin of the wearer.

The raised structure 160 is configured to surround the opening 165, where the LEDs of the PPG sensor module(s) 455 are aligned to emit light through the opening 165 and the raised structure 160 partially enters and is attached to portion into the third cavity 340 to prevent unwanted movement of the PPG sensor module(s) 455. The raised structure 160 is further configured to exclude detection of light which has only reflected from the skin surface and i.e., to restrict light piping.

Third and fourth raised structures 156 and 158 are coupled to the raised structure 160. Each of these raised structures 152, 154, 156, 158 and 160 is formed on the top side 162 of the second housing 165 to secure the components of the biosensing logic 130 from unwanted movement. Also, when the second housing 150 is attached to the first housing 110, the raised structures 152, 154, 156, 158 and 160 are configured to seal components of the biosensing logic 130 within their respective cavities.

Additionally, the second housing 150 features the bottom surface 163 that, when the second housing 150 is attached to the first housing 110 as shown in FIG. 10, is generally coplanar with bottom surfaces associated with the first housing 110. This coplanar configuration allows for better (or at least more universal) adhesion the wearable biosensing device 100 to the wearer.

Referring now to FIG. 7, a bottom perspective view of the first housing 110 for the wearable biosensing device 100 of FIG. 2 is shown. Herein, an underside 600 of the base 205 of the first housing 110 is shown. The underside 600 features the first and second bottom surfaces 127 and 128 of the base 205 being part of the first housing 110. The underside 600 further features cavities 300, 310, 340, 350 and 360 positioned between the first and second bottom surfaces 127 and 128.

According to one embodiment of the disclosure, the first cavity 300 includes the additional sub-cavities 370, which are arranged to provide sufficient clearance for components that are placed within the cavity such as, for example, the processing logic 410, the memory logic 420, and the communication logic 430 mounted on the substrate 400 forming the electronics assembly 135 of FIG. 2. The third cavity 310 constitutes a structural tier-based cavity, which provides sufficient clearance for components of the power assembly 145. The second cavity 340 provides sufficient clearance for the components associated with the sensing assembly 140. The fourth and fifth cavities 350 and 360 provide sufficient clearance for the interconnects 490 and 495, respectively.

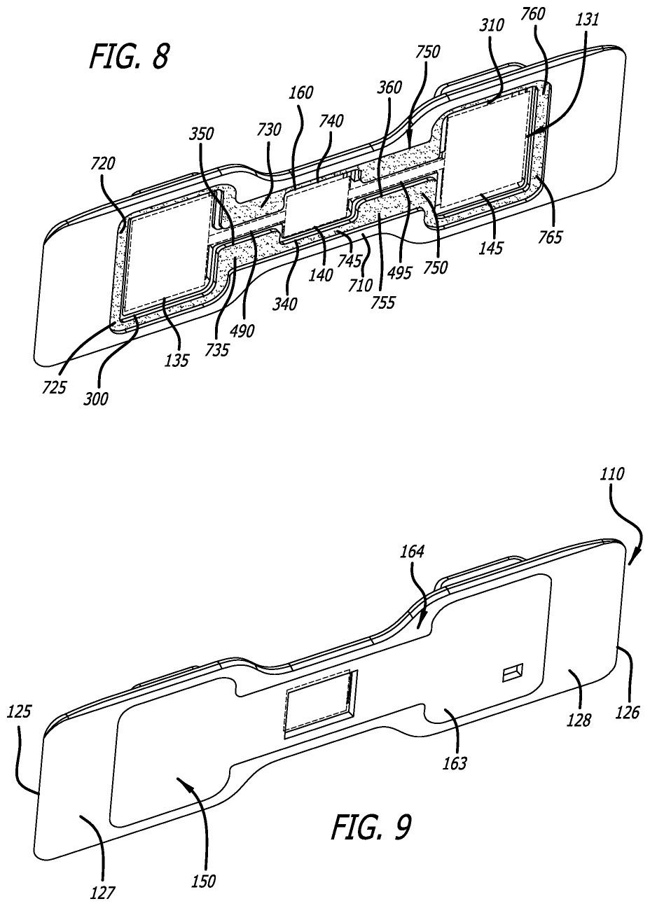

Referring to FIG. 8, a bottom perspective view of the first housing 110 for the wearable biosensing device 100 of FIG. 2 is shown, where the biosensing logic 130 of FIG. 5 is deployed within cavities 300, 310, 340, 350 and 360 formed within the first housing 110. Herein, as shown in both FIGS. 7-8, an adhesive 700 is attached to interior, recessed surfaces 710 of the first housing 110. As shown for this embodiment, the interior, recessed surfaces 710 includes a first recessed surface 720, a second recessed surface 730, a third recessed surface 740, a fourth recessed surface 750, and a fifth recessed surface 760.

The first recessed surface 720 is formed around a periphery of the first cavity 300 and intersects with the second recessed surface 730. The second recessed surface 730 extends along the fourth cavity 350 operating as a channel for the first interconnect 490. The second recessed surface 730 intersects with the third recessed surface 740. The third recessed surface 740 is formed along an outer side of the raised structure 160 and extends to intersect with the fourth recessed surface 750. The fourth recessed surface 750 extends along the fifth cavity 360 operating as a channel for the second interconnect 495 before intersecting with the fifth recessed surface 760. The fifth recessed surface 760 is formed around a periphery of the second cavity 310. Complementary recessed surfaces 725, 735, 745, 755 and 765 are positioned along an opposite longitudinal side of the cavities 300, 310, 340, 350 and 360.

Referring to FIG. 9, a bottom perspective view of the wearable biosensing device 100 is shown, illustrating the second housing 150 being coupled to the first housing 110 to encapsulate the biosensing logic 130. Herein, the second housing 150 is attached to the first housing 110 by insertion of the raised structures 152, 154, 156, 158 and 160 (see FIGS. 1, 5) partially into cavities 300, 310, 340, 350 and 360, respectively. As a result, the bottom surface 163 of the second housing 150, after attachment of the second housing 150 to the first housing 110, is generally coplanar with bottom surfaces 127 and 128 of the first and second end portions 125 and 126 of the first housing 110, respectively. According to this embodiment of the disclosure, the bottom surface 163 of the second housing 150 may be coplanar to bottom surfaces 164 of at least areas within the first housing 110 occupied by the deformable stiffening element 220 of FIG. 3. As a result, according to one embodiment of the disclosure, the entire bottom surface 163 of the second housing 150 and certain areas of the bottom surface 164 of the housing 110 are substantially coplanar.

Referring now to FIG. 10, a bottom-side perspective view of the wearable biosensing device 100 is shown, Herein, the adhesive layer 170 is applied to bottom surface 163 of the second housing 150 as well as the bottom surfaces 127 and 128 of the first and second end portions 125 and 126 of the first housing 110. The adhesive layer 170 is applied so that a presence of adhesive 900 extends from the bottom surface 127 of the first end portion 125, over the bottom surface 163 of the second housing 150, and ceases at the bottom surface 128 of the second end portion 126 of the first housing 110. As shown, the backside co-planar arrangement assists in the application of the wearable biosensing device 100 to the wearer.

Referring to FIG. 11, a bottom, cut-away view of the wearable biosensing device 100 is shown. The wearable biosensing device 100 includes the first housing 110 featuring the deformable stiffening element 220, the biosensing logic 130, the second housing 150, and the centrally located opening 165 for positioning of the optical sensor(s) 450 (e.g., different LEDs 4521-4523, complementary photodetectors 4541-4543) deployed within the shielding member 445 of the sensing assembly 140 positioned above the opening 165.

### IV. Functionality Representation of the Wearable Biosensing Device

Herein, the first housing 110, the second housing 150 and the biosensing logic 130 are linearly elastic and do not deform plastically in intended use cases. For a linearly elastic, simply supported beam the bending force in response to an external load is given by equation (1), where “F” is the applied load, “E” is the modulus of elasticity for the material, “I” is the area moment of inertia, “l” is the length at which the load is acting, and “8” is the displacement:

$$F = \frac{48 EI}{{l}^{3}} δ \qquad equation ( 1 )$$

Herein, equation (1) is analogous to Hooke's law, F=kδ where k=48EIl⁻³ is representative of a “spring” constant. Herein, the first housing 110, the second housing 150 and the biosensing logic 130 of FIG. 1 are laminated together, which implies all displacements are common at any point of the collective component. Therefore, the wearable biosensing device 100, without the deformable stiffening element 220, can be modeled as a system of springs in parallel as shown in FIG. 12A, where the stiffness of the wearable biosensing device 100 may constitute a sum of component stiffness k_{c} 1200. For this illustrative embodiment, the combined stiffness k_{c} may constitute the sum of the stiffness 1210 of the first housing (kₜₕ), the stiffness 1220 of the second housing 150 (k_{bh}), and the stiffness 1230 of the biosensing logic (K_{PCBA}).

With respect to the wearable biosensing device 100 of FIGS. 2-3, the deformable stiffening element 220 has been added to balance the elastic force, enabling the wearable biosensing device 100 to conform to fistula anatomy. According to one embodiment of the disclosure, the deformable stiffening element 220 comprises 1100 series aluminum, where the stresses induced in the deformable stiffening element 220 during plastic deformation are constant during, and thus, the wearable biosensing device 100 can be modeled by a spring of infinite stiffness (does not displace) in series with a prismatic joint, leading to a constant force F_{y}. Hence, the deformable stiffening element 220 also experiences the same deformation as the rest of the device assembly, which implies that the deformable stiffening element 220 and by extension the prismatic joint are in parallel with the combined stiffness of the first and second housings 110 and 150 as well as the biosensing logic 130. Herein, as shown in FIG. 12A, the stiffening yield force (Fy) 1240 associated with the deformable stiffening element 220 (F_{y}≥δk_{c} and also F_{y}≈δk_{c}) is slight greater than the bending force (F_{b}). Stated differently, the force required to yield the stiffening element 220 (F_{y}) 1240 is greater than the elastic forces (δk_{c}) applied by the rest of the components of the wearable biosensing device 100. This leads to desired behavior that the deformation of the wearable biosensing device 100 is nearly exclusively plastic, which has the effect of limiting the forces that the adhesive must support, thereby extending adhesive life.

In summing the forces in FIG. 12B, the results in the formation of below-listed equation (2) are computed. In a completely static scenario, the combined (adhesive) forces F_{A} 1250, the forces applied by the device housings 110/150 and biosensing logic 130 are balanced by the stiffening yield force (Fy) 1240 provided by the stiffening element 220 so that the adhesive force (F_{A}) is negligible (F_{A}=0). In a dynamic situation F_{y}≈F_{b}=k_{c}δ, which implies that adhesive force F_{A} is small.

$${F}_{A} = {F}_{b} - {F}_{y} \qquad equation ( 2 )$$

In the foregoing description, the invention is described with reference to specific exemplary embodiments thereof. Hence, it will be evident that certain components may be deployed within different types of wearable biosensing devices and various modifications and changes may be made thereto without departing from the broader spirit and scope of the invention as set forth in the appended claims.

What is claimed is:

1. A wearable biosensing device, comprising:
a first housing including a housing element and a deformable stiffening element inserted within the housing element, the housing element includes a plurality of lobes; and
a biosensing logic encapsulated within the plurality of lobes, wherein an electronics assembly is positioned within a first lobe of the plurality of lobes, a sensing assembly is positioned within a second lobe of the plurality of lobes, and an interconnect for communicative coupling of the electronics assembly and the sensing assembly,
wherein forces applied by at least the first housing and the biosensing logic are balanced by the deformable stiffening element.

2. The wearable biosensing device of claim 1, wherein the deformable stiffening element is molded as part of the housing element.

3. The wearable biosensing device of claim 1, wherein the deformable stiffening element is positioned along widthwise and lengthwise perimeters of at least the first lobe and a third lobe of the plurality of lobes, the third lobe encapsulates a power assembly including a substrate, power management logic mounted on the substrate, and power supply logic mounted on the substrate.

4. The wearable biosensing device of claim 1, wherein the deformable stiffening element is positioned within a continuous, recessed portion of the housing element.

5. The wearable biosensing device of claim 1, wherein the deformable stiffening element comprises (1) a first longitudinal edge including a first segment, a second segment and a third segment interposed between the first segment and the second segment and (2) a second longitudinal edge including a fourth segment, a fifth segment and a sixth segment interposed between the four segment and the fifth segment, where a distance between the first segment of the first longitudinal edge and the fourth segment of the second longitudinal edge is greater than a distance between the second segment of the first longitudinal edge and the fifth segment of the second longitudinal edge.

6. The wearable biosensing device of claim 1 further comprising a second housing including an opening for alignment with optical sensors and photodetectors deployed as part of the sensing assembly of the biosensing logic.

7. The wearable biosensing device of claim 6, wherein the optical sensors and photodetectors are aligned with the opening to emit and detect light reflecting or refracting from a vessel to detect properties of the vessel and biological fluid flow through the vessel.

8. The wearable biosensing device of claim 7, wherein the vessel corresponds to an arteriovenous (AV) fistula.

9. The wearable biosensing device of claim 8, wherein the sensing assembly includes audio sensing logic.

10. The wearable biosensing device of claim 1, wherein the sensing assembly includes audio sensing logic, the audio sensing logic comprises a microphone.

11. A wearable biosensing device, comprising:
a first housing including a housing element and a deformable stiffening element inserted within the housing element, the housing element includes a plurality of lobes formed with a corresponding plurality of cavities, wherein a first lobe being formed to include a first cavity, a second lobe being formed to include a second cavity, and a third lobe being formed to include a third cavity; and
a biosensing logic inserted into the plurality of cavities, wherein an electronics assembly is positioned within the first cavity of the plurality of cavities, a sensing assembly is positioned within the second cavity of the plurality of cavities, and an interconnect for communicative coupling of the electronics assembly and the sensing assembly is positioned within the third cavity of the plurality of cavities.

12. The wearable biosensing device of claim 11, wherein the deformable stiffening element is molded as part of the housing element.

13. The wearable biosensing device of claim 11, wherein the deformable stiffening element is positioned along widthwise and lengthwise perimeters of at least the first lobe and a fourth lobe with a fourth cavity, the fourth cavity includes a power assembly communicatively coupled to the sensing assembly via a second interconnect, the power assembly comprises a substrate, power management logic mounted on the substrate, and power supply logic mounted on the substrate.

14. The wearable biosensing device of claim 11, wherein the deformable stiffening element is positioned within a continuous, recessed portion of the housing element.

15. The wearable biosensing device of claim 11, wherein the deformable stiffening element comprises (1) a first longitudinal edge including a first segment, a second segment and a third segment interposed between the first segment and the second segment and (2) a second longitudinal edge including a fourth segment, a fifth segment and a sixth segment interposed between the four segment and the fifth segment, where a distance between the first segment of the first longitudinal edge and the fourth segment of the second longitudinal edge is greater than a distance between the second segment of the first longitudinal edge and the fifth segment of the second longitudinal edge.

16. The wearable biosensing device of claim 11 further comprising a second housing including an opening for alignment with audio sensing logic, optical sensors and photodetectors deployed as part of the sensing assembly of the biosensing logic.

17. The wearable biosensing device of claim 16, wherein the optical sensors and photodetectors are aligned with the opening to emit and detect light reflecting or refracting from a vessel to detect properties of the vessel and biological fluid flow through the vessel.

18. The wearable biosensing device of claim 17, wherein the vessel corresponds to an arteriovenous (AV) fistula.

19. The wearable biosensing device of claim 11, wherein the sensing assembly includes audio sensing logic, the audio sensing logic comprises a microphone.

20. A wearable biosensing device, comprising:
one or more housings including a first housing including a housing element and a deformable stiffening element inserted within the housing element, the housing element includes a plurality of lobes; and
biosensing logic encapsulated within the plurality of lobes, the biosensing logic comprises
an electronics assembly positioned within a first lobe of the plurality of lobes,
a sensing assembly positioned within a second lobe of the plurality of lobes, the sensing assembly communicatively coupled to the electronics assembly via a first interconnect, and
a power assembly positioned within a third lobe of the plurality of lobes, the power assembly communicatively coupled to the sensing assembly via a second interconnect,
wherein forces applied by at least the one or more housings and the biosensing logic are balanced by the deformable stiffening element.
